(19) United States
(12) Patent Application Publication
(10) Pub. No.: US 2015/0132731 A1
(43) Pub. Date: May 14, 2015

(54) PHYSICAL THERAPY SYSTEM

(71) Applicant: HEALTH TECH PAL CORP, Dallas, TX (US)

(72) Inventor: NAVEEN KHAN, LOS ANGELES, CA (US)

(21) Appl. No.: 14/539,356

(22) Filed: Nov. 12, 2014

Related U.S. Application Data
(60) Provisional application No. 61/903,090, filed on Nov. 12, 2013.

(51) Int. Cl. G09B 5/00; G09B 23/28

(52) U.S. Cl. CPC G09B 5/00; G09B 23/28; A61B 2505/09
USPC 434/262

(57) ABSTRACT

A technique of attempting to help a person successfully complete physical therapy. The technique includes at least the following steps: providing, via a first computing device including at least tangible computing elements, options for one or more physical therapy exercises to the first person; providing, via the first computing device, instructions for performing the physical therapy exercises to the first person; receiving, by the first computing device, information from the first person about performance of the physical therapy exercises; and transmitting the information from the first computing device to a second computing device used by a second person. Also, systems that perform the technique.

### CROSS-REFERENCE TO RELATED APPLICATIONS

This application claims the benefit of U.S. Provisional Application No. 61/903,090, filed on Nov. 12, 2013, and entitled PHYSICAL THERAPY SYSTEM, which is incorporated herein in its entirety.

### BACKGROUND

The present disclosure generally relates to a system intended to help people successfully complete physical therapy.

### SUMMARY

Aspects of the subject technology include a technique of attempting to help a person successfully complete physical therapy. The technique includes at least the following steps: providing, via a first computing device including at least tangible computing elements, options for one or more physical therapy exercises to the first person; providing, via the first computing device, instructions for performing the physical therapy exercises to the first person; receiving, by the first computing device, information from the first person about performance of the physical therapy exercises; and transmitting the information from the first computing device to a second computing device used by a second person. The subject technology also includes systems configured to perform the above technique.

This brief summary has been provided so that the nature of the invention may be understood quickly. Additional steps and/or different steps that those set forth in this summary may be used. A more complete understanding of the invention may be obtained by reference to the following description in connection with the attached drawings.

### BRIEF DESCRIPTION OF THE DRAWINGS

For a more complete understanding, reference is now made to the following description taken in conjunction with the accompanying Drawings in which:

FIG. 1 illustrates an environment in which a system attempts to help a person successfully complete physical therapy according to aspects of the subject technology; and

FIGS. 2 to 21 illustrate screenshots that may be used by a system that attempts to help a person successfully complete physical therapy according to aspects of the subject technology.

### DETAILED DESCRIPTION

Referring now to the drawings, wherein like reference numbers are used herein to designate like elements throughout, the various views and embodiments of a physical therapy system are illustrated and described, and other possible embodiments are described. The figures are not necessarily drawn to scale, and in some instances the drawings have been exaggerated and/or simplified in places for illustrative purposes only. One of ordinary skill in the art will appreciate the many possible applications and variations based on the following examples of possible embodiments.

Briefly, techniques according to aspects of the subject technology include a system that attempts to help a person successfully complete physical therapy. One problem that often arises with physical therapy is that patients often do not perform their exercises properly. A patient may not perform an exercise in the correct manner, may not perform the correct number of repetitions or sets, may skip exercises, or may otherwise fail to perform the exercises properly. The results of failing to perform the exercises may be dire, up to and including permanent loss of range of motion (e.g., frozen shoulder, hip, knee, or other joint) or the need for surgery to correct such loss of range of motion. In some cases, even surgery may not be able to fully restore range of motion.

One manner of addressing the foregoing issues is to have a patient visit a physical therapist each time the patient is supposed to perform physical therapy exercises. The therapist may then ensure that the patient performs the exercises properly. However, visiting a physical therapist every time the exercises need to be performed may be time consuming and costly. Furthermore, a physical therapist may only be able to see a limited number of patients if the therapist has to supervise each patient during the patient's entire course of therapy.

Aspects of the subject technology attempt to address the foregoing issues. In one aspect, a system that includes at least a first computing device having at least tangible computing elements is provided. The first computing device may be used by a physical therapy patient. The computing elements may perform steps that include at least the followings: providing options for one or more physical therapy exercises to the first person; providing instructions for performing the physical therapy exercises to the first person; receiving information from the first person about performance of the physical therapy exercises; and transmitting the information from the first computing device to a second computing device used by a second person. The second computing device may be used by a physical therapist.

The computing devices may be mobile computing devices such as smart phones, laptop computers, desktop computers, or any other type of computing devices that include at least tangible computing elements. The information may be transmitted from the first computing device to the second computing device through one or more other computing devices, one or more networks, or both. For example, the information may be transmitted from the first computing device to the second computing device via the Internet.

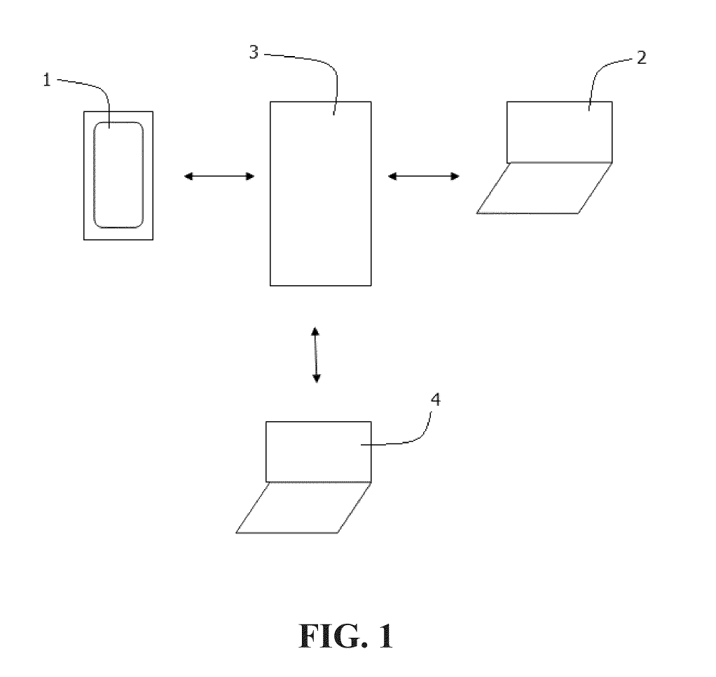

FIG. 1 illustrates an environment in which such a system may operate. A first computing device is shown in the form of smart phone 1. This computing device may be used to provide options for one or more physical therapy exercises to a physical therapy patient, to provide instructions for performing the physical therapy exercises to the patient, and to receive information from the patient about performance of the physical therapy exercises. This information may then be transmitted from the first computing device to a second computing, which is illustrated in the figure as laptop 2. The second computing device may be used by a therapist to monitor the patient's performance of the exercises.

The computing devices are shown communicating via another computing device such as a server 4 over a network, for example via a web portal running on a web server. The first and second computing devices may communicate via any other type of computing device(s) and/or networks(s) or the device may communicate directly with each other.

Also illustrated in FIG. 1 is a third computing device in the form of another laptop computer 4. This computing device may be used by an administrator to set up access to and use of the subject technology by patients and/or therapists. Other types of computing devices may be used to perform this function.

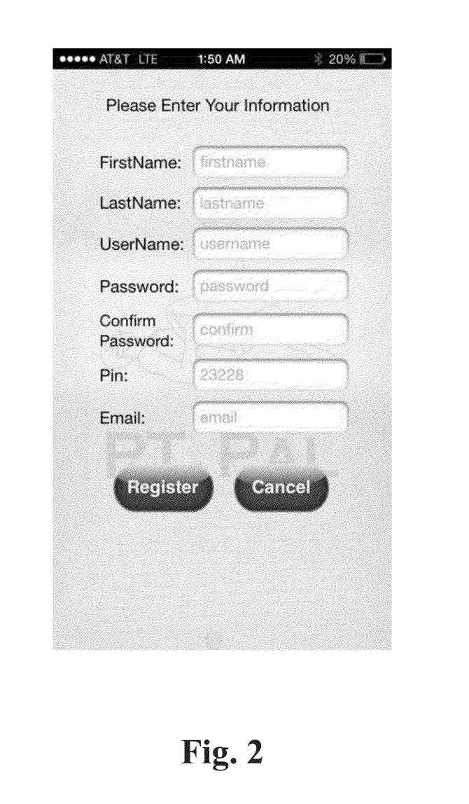

FIGS. 2 to 21 illustrate screenshots that may be used by a system that attempts to help a person successfully complete physical therapy according to aspects of the subject technology. Screenshots for display to a patient, a therapist, and an administrator are illustrated. The screenshots in FIGS. 2 to 21 illustrate aspects of the subject technology in which the patient uses an app running on a smart phone and the therapist uses a laptop or desktop computer to interact via a web portal. The administrator also interacts with the web portal via a laptop or desktop computer. The subject technology is not limited to these screenshots, which illustrate one of many possible ways of implementing the subject technology.

FIG. 2 illustrates a screenshot for a patient to register their details via an app running on a smart phone. In some aspects, a therapist, trainer, or some other person may provide a personalized pin code so that the patient can be registered on a web portal via the app.

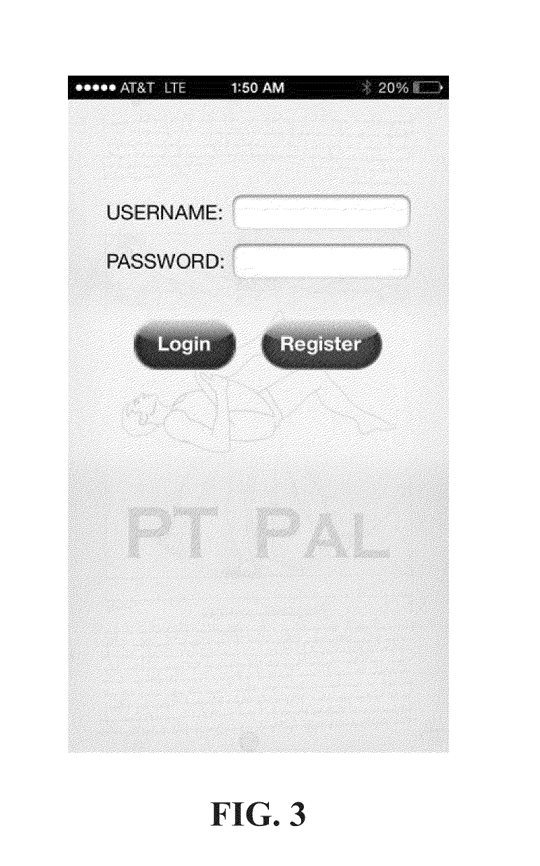

If a patient is already registered, the patient may be requested to log in using a username and password as illustrated in FIG. 3.

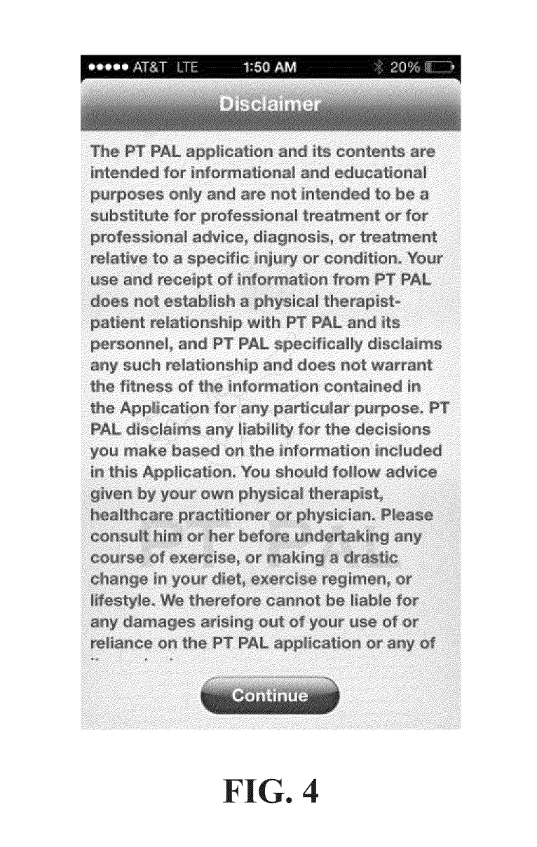

In some aspects, the patient is required to agree to a disclaimer as illustrated in FIG. 4 before using the app.

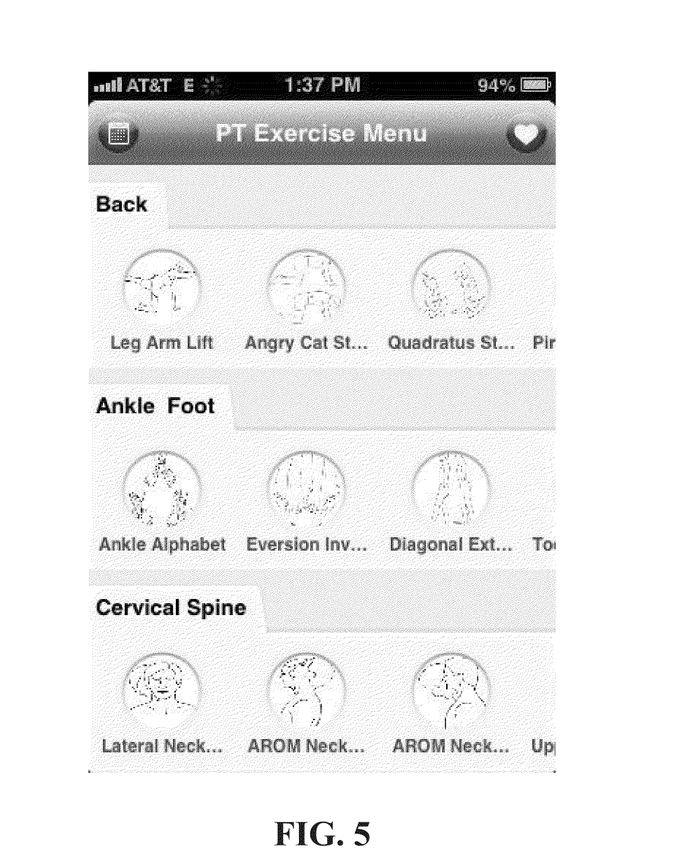

FIG. 5 illustrates a Main Menu. Available exercises in different categories corresponding to different body parts are accessible. Different categories may be seen by scrolling up and down, and different exercises within the categories may be seen by scrolling left and right. Alternatively, categories and exercises may be arranged and/or accessed in different ways.

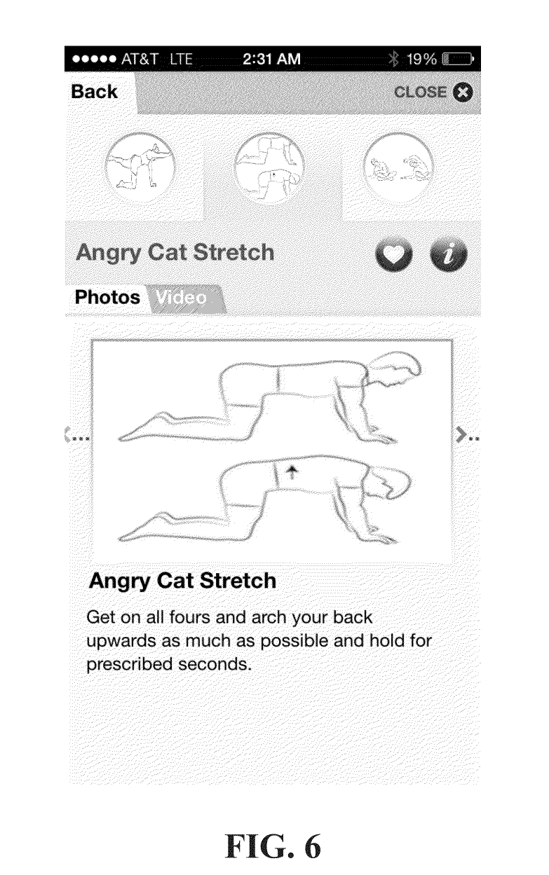

A patient may touch an exercise shown in the Main Menu to view instructions for that exercise. Instructions may be viewed in various forms. Examples illustrated in FIG. 6 include written instructions, a drawing, photos, and video. Instructions for different exercises within the current exercise's category may be accessed by scrolling the exercises at the top of the screenshot. A patient may select an exercise as a “favorite,” that is an exercise that the patient will perform, by touching the heart. If an exercise has already been selected as a favorite, the heart may be “greyed out” to so indicate.

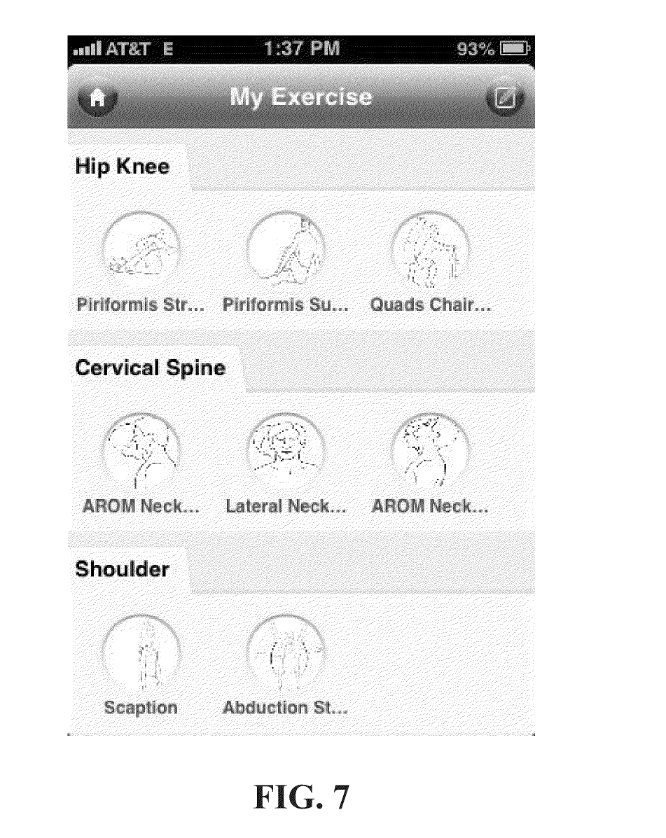

After “Close” is selected on the screenshot illustrated in FIG. 6, the Main Menu may be shown. If the patient touches the heart on this screen, the exercises that the patient has selected as “favorites” may be shown as illustrated in FIG. 7.

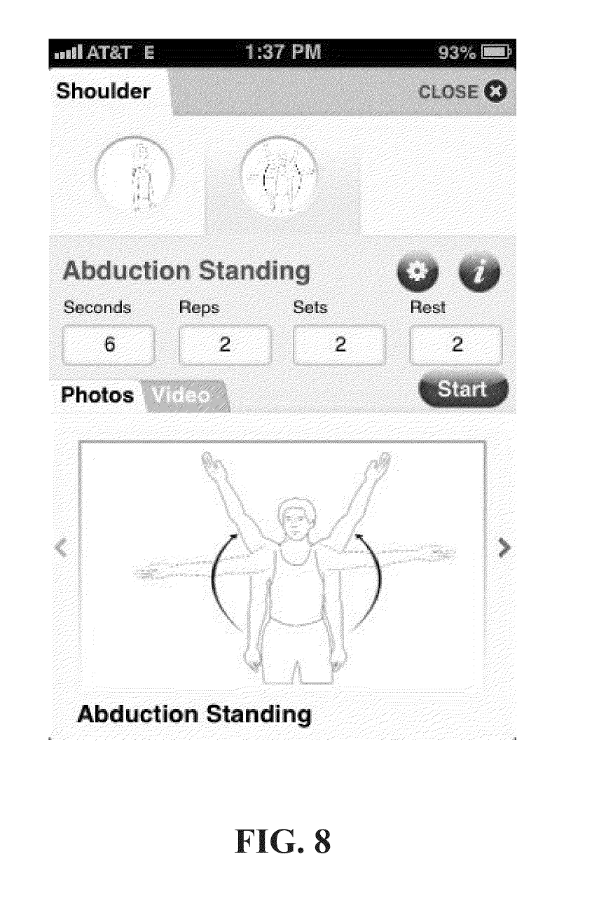

The screenshot in FIG. 8 illustrates a personalized menu from which the patient's exercises may be accessed by scrolling at the top of the screen. This screen may be accessed by selecting one of the exercises in FIG. 7. Options for each exercise may be shown. In the example illustrated in FIG. 8, the options include a time for each repetition of the exercise, a number of repetitions in a set for the exercise, a number of sets for the exercise, and a rest time between each of the sets for the exercise. These options, other options, or some combination thereof may be shown. Preferably, the patient may edit these options. When “Start” is chosen, instructions may be given for the exercise, for example “start,” “rest” and “finish” as seconds for performing the exercise counts down. In some aspects, if patient starts an exercise, it may be assumed that the person completed the exercise unless the patient indicates otherwise. In other aspects, a button may be provided for the patient to indicate that he or she completed the exercise.

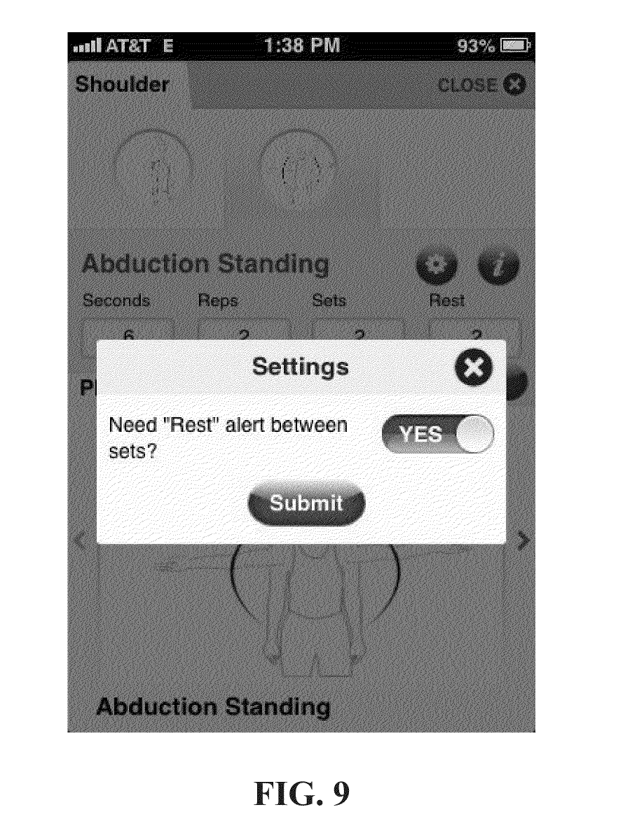

A settings button (i.e., the gear shaped button) is also illustrated in FIG. 8. An example of a setting is illustrated in FIG. 9, namely an option to have a “rest” alert given between sets.

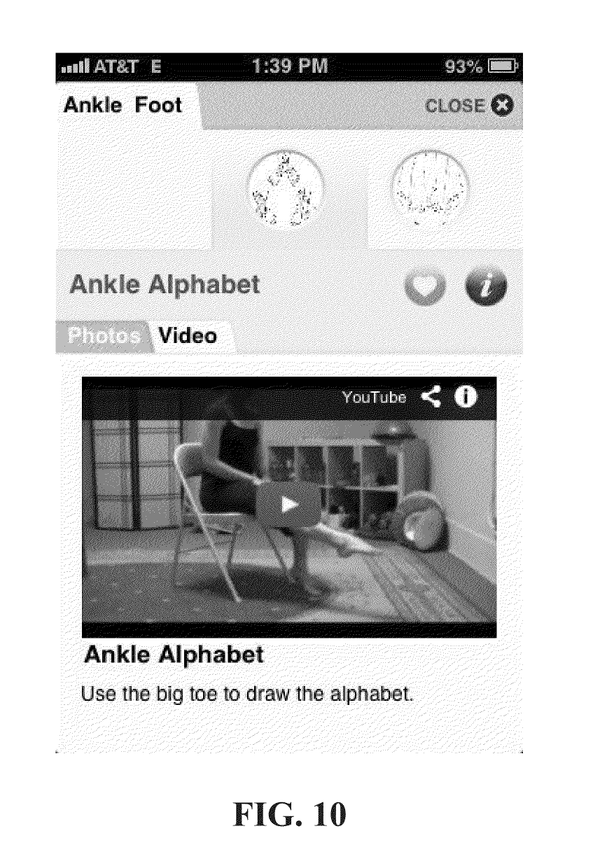

The screenshot in FIG. 8 also includes “Photo” and “Video” tabs. These tabs permit the patient to access photo and/or video instructions for the exercise, for instructional replay later, for example as illustrated in FIG. 10.

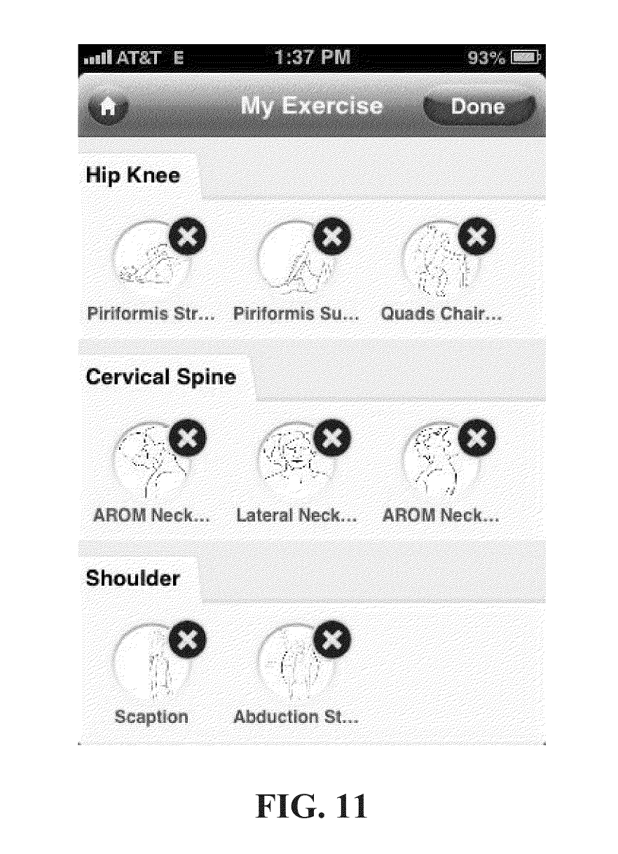

FIG. 11 illustrates the “My Exercise” screenshot in edit mode. This mode may permit a patient to delete one or more exercises from the patient's favorites. The screen may be accessed by holding down on any of the exercises in the “My Exercise” screenshot illustrated in FIG. 8 for about one second.

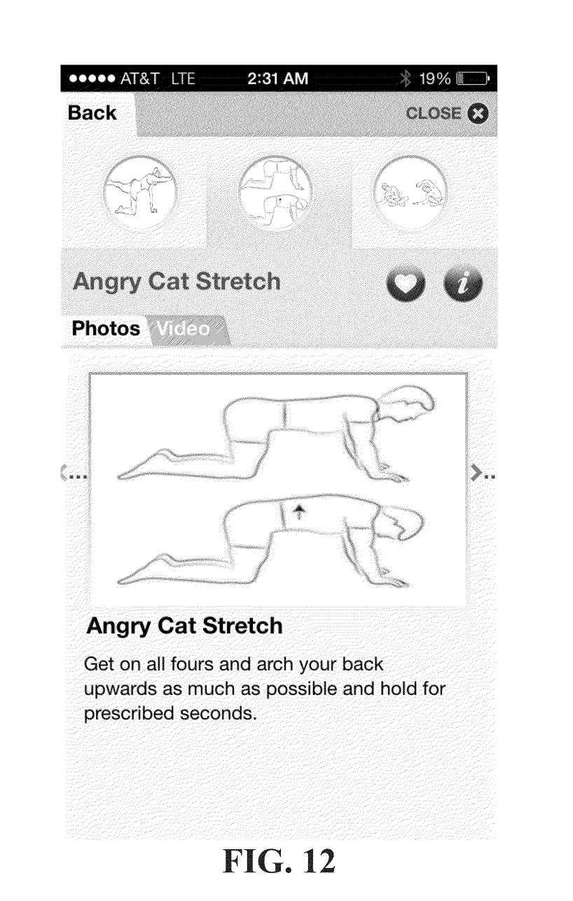

As mentioned above, the patient may access their exercises by scrolling at a top of the screenshot illustrated in FIG. 8. FIG. 12 illustrates showing “Angry Cat Stretch” after such scrolling has occurred.

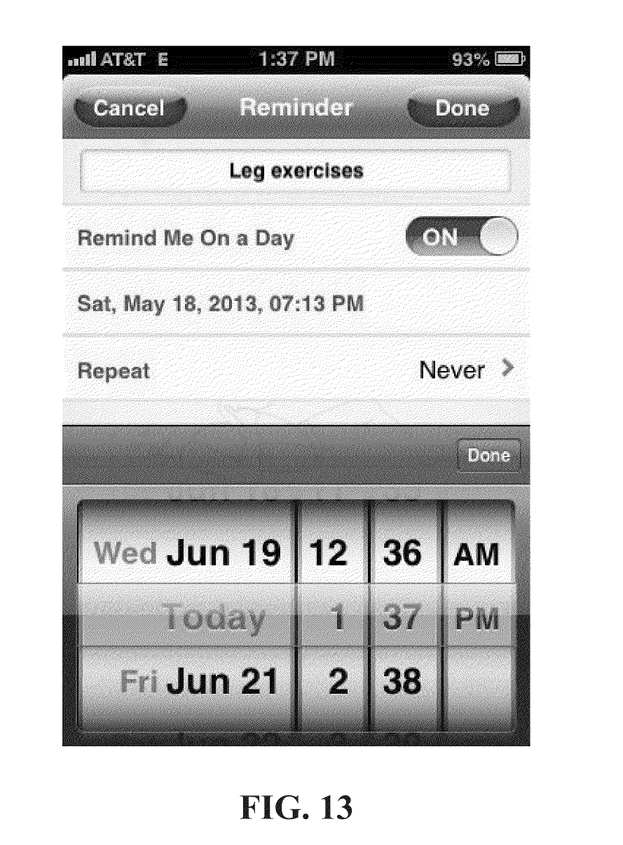

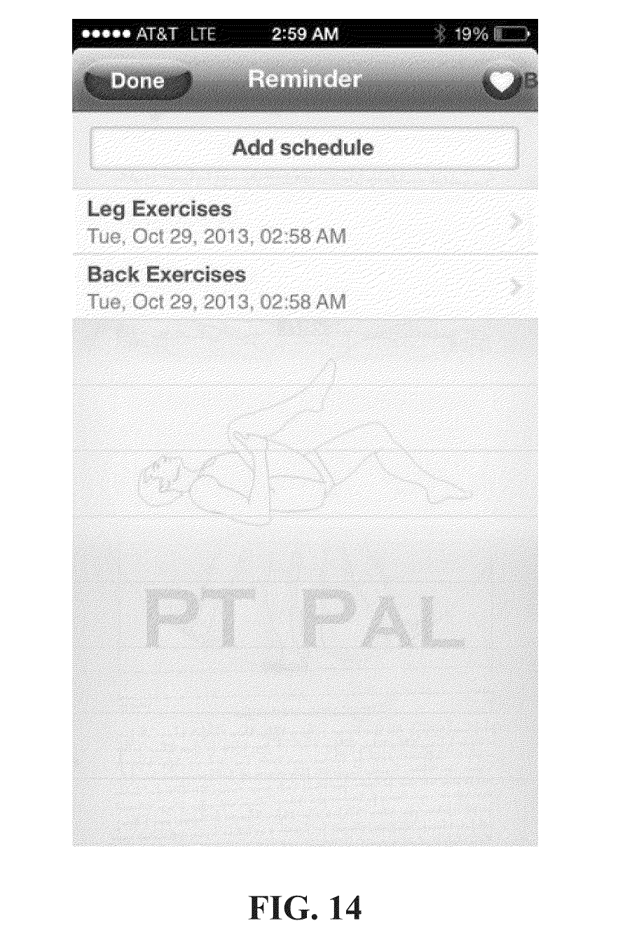

The patient may schedule reminders to do the patient's exercises. A screenshot of an example for scheduling such reminders is illustrated in FIG. 13. As many reminders as desired (within technical limitations) may be scheduled. Two scheduled reminders are illustrated in FIG. 14.

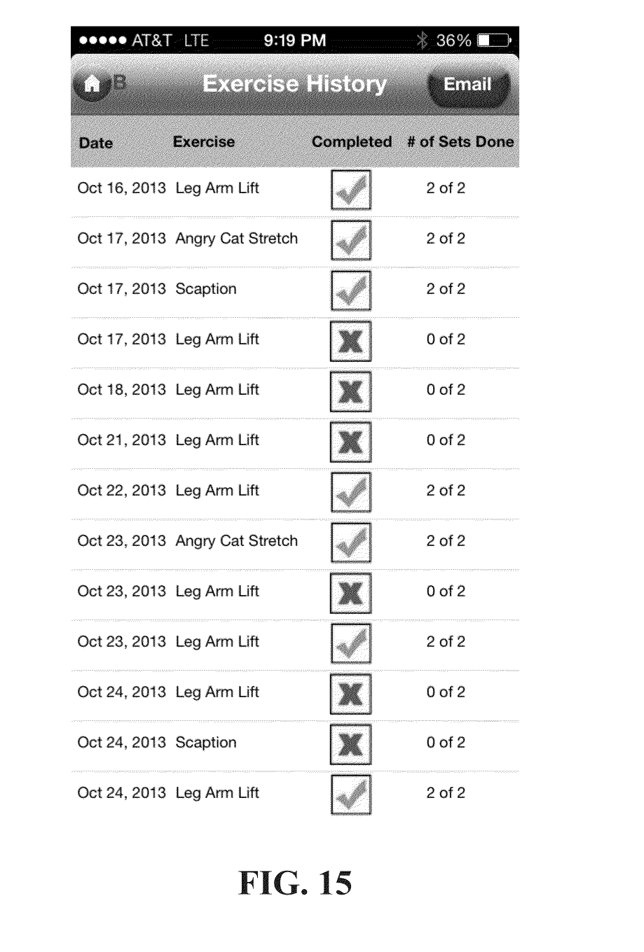

FIG. 15 illustrates a screenshot showing the patient's exercise history, preferably including indications of when the patient successfully performed and failed to perform the scheduled exercises. Preferably, the history is automatically updated by the app and is available via a web portal in real time.

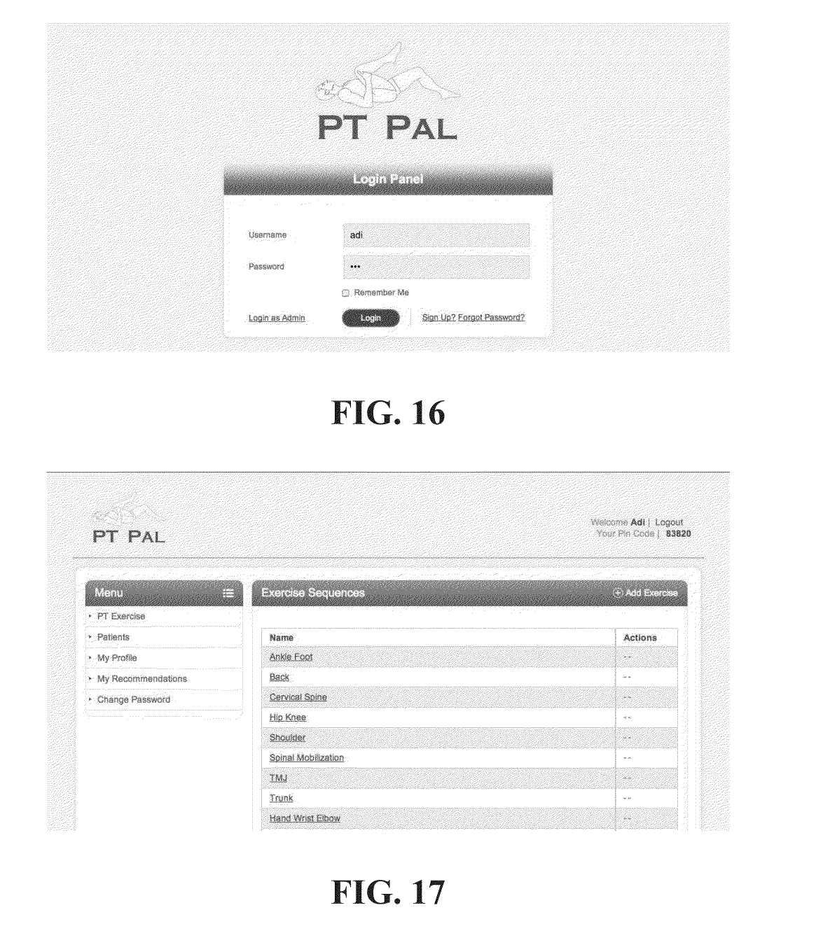

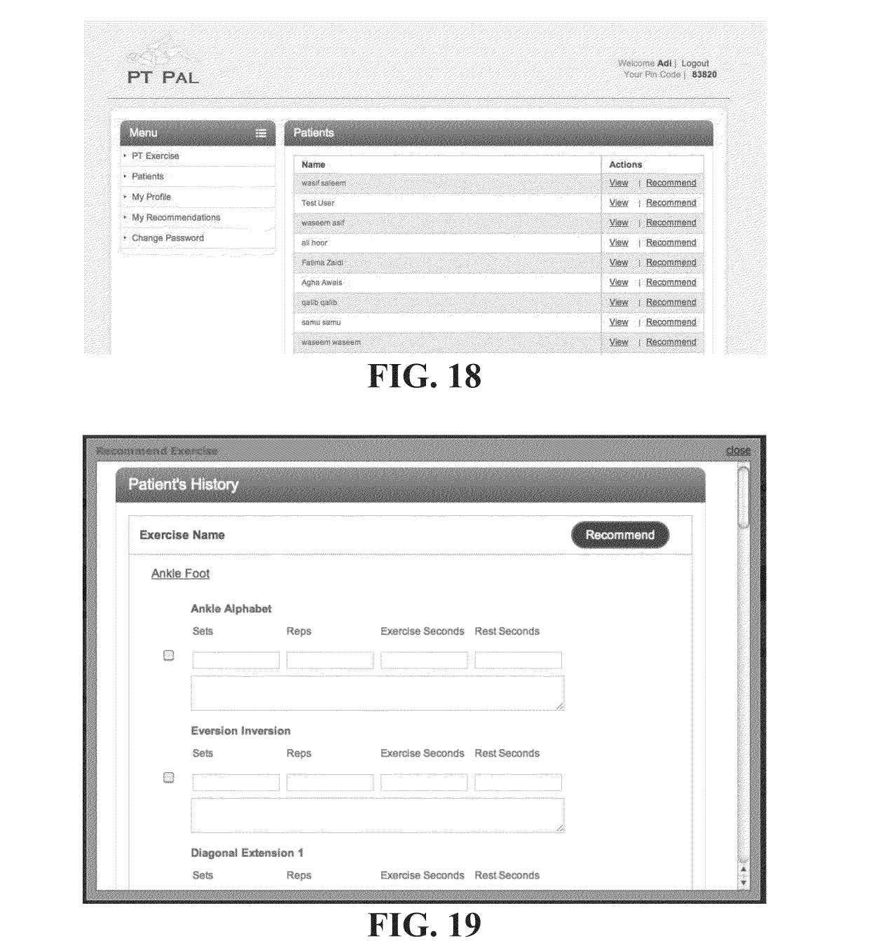

FIG. 16 illustrates a screenshot that a physical therapist may use to log into the web portal, for example using a laptop, desktop, or other computing device. FIG. 17 illustrates that the therapist may see a list of patients whose physical therapy the therapist is overseeing. The therapist may send a patient a recommended exercise that the person should perform, as illustrated in FIG. 18. The recommend exercise may automatically appear on the patient's “My Exercise” screen or the patient may have to approve inclusion of the exercise.

The therapist may view a patient or client's exercise history, as illustrated in FIG. 19. As noted above, the patient's exercise history preferably is updated and therefore accessible in real time. Thus, a therapist may use aspects of the subject technology to attempt to ensure that his or her patients are completing their physical therapy exercises properly without the patients visiting the therapist in person. As a result, a therapist may be able to provide services in a more effective manner to more patients. Of course, in-person visits may also be part of the physical therapy program.

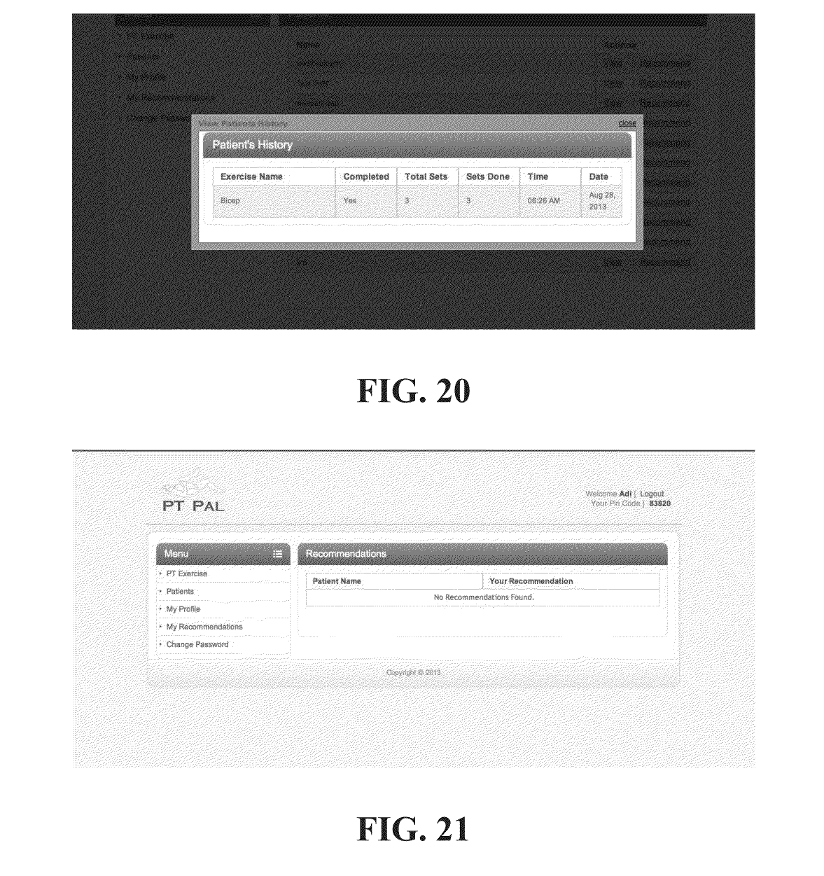

FIG. 20 illustrates an additional screenshot that shows a history of exercises that the therapist has recommended. This history may be combined with tracking patient progress to craft more effective courses of exercises for physical therapy.

FIG. 21 illustrates a screenshot of an administration page by which an administrator may manage patient profiles, therapist profiles, assignment of patients to therapists, recommendations, and/or the like.

The invention is in no way limited to the specifics of any particular embodiments and examples disclosed herein. For example, the terms “aspect,” “example,” “preferably,” “alternatively” and the like denote features that may be preferable but not essential to include in some embodiments of the invention. In addition, details illustrated or disclosed with respect to any one aspect of the invention may be used with other aspects of the invention. Additional elements and/or steps may be added to various aspects of the invention and/or some disclosed elements and/or steps may be subtracted from various aspects of the invention without departing from the scope of the invention. Singular elements/steps imply plural elements/steps and vice versa. Steps may be performed serially, in parallel, in a pipelined manner, or in different orders than disclosed herein. Many other variations are possible which remain within the content, scope and spirit of the invention, and these variations would become clear to those skilled in the art after perusal of this application.

In one aspect, a system is included that attempts to help a first person successfully complete physical therapy, comprising a first computing device including at least tangible computing elements that perform steps comprising providing options for one or more physical therapy exercises to the first person; providing instructions for performing the physical therapy exercises to the first person, receiving information from the first person about performance of the physical therapy exercises, and transmitting the information from the first computing device to a second computing device used by a second person.

Further, the system includes options for the physical therapy exercises comprising selecting from among possible exercises.

Further, the system includes the possible exercises being arranged for selection in categories.

Further, the system includes each of the categories containing exercises for a particular body part.

Further, the system includes the options for a physical therapy exercise comprising one or more of time for each repetition of the exercise, a number of repetitions in a set for the exercise, a number of sets for the exercise, and a rest time between each of the sets for the exercise.

Further, the system includes the steps further comprising recording the information about the performance of the physical therapy exercises over time.

Further, the system includes the steps further comprising providing the information about the performance of the physical therapy exercises over time to the first person.

Further, the system includes the step of transmitting the information from the first computing device to the second computing device further comprises transmitting the information about the performance of the physical therapy exercises over time to the second computing device.

Further, the system includes the information being transmitted from the first computing device to the second computing device through one or more other computing devices, one or more networks, or both.

Further, the system includes the information being transmitted from the first computing device to the second computing device via the Internet.

It should be understood that the drawings and detailed description herein are to be regarded in an illustrative rather than a restrictive manner, and are not intended to be limiting to the particular forms and examples disclosed. On the contrary, included are any further modifications, changes, rearrangements, substitutions, alternatives, design choices, and embodiments apparent to those of ordinary skill in the art, without departing from the spirit and scope hereof, as defined by the following claims. Thus, it is intended that the following claims be interpreted to embrace all such further modifications, changes, rearrangements, substitutions, alternatives, design choices, and embodiments.

What is claimed is:

1. A method for effectively implementing a therapist defined therapeutic regimen and a patient, comprising the steps of:
providing a central processing system for storing configuration information, status information and patient profile information associated with a plurality of therapist defined therapeutic regimens;
providing a therapist computer interface for allowing a therapist to interface with the central processing system to define therapeutic regimens for a patient and monitor such;
providing a patient computing device with a display for allowing a patient to interface with the central processing system for running a personal therapeutic application thereon for allowing the patient to implement a therapist defined therapeutic regimen for the patient;
configuring by the therapist via the therapist computer interface the therapist defined therapeutic regimen for the patient at the central processing system;
transferring the configured therapist defined therapeutic regimen for the patient to the patient computing device after configuration;
executing by the patient the configured therapist defined therapeutic regimen for the patient by the steps of:
selecting the configured therapist defined therapeutic regimen for the patient that was transferred to the patient's patient computing device;
selecting from the selected configured therapist defined therapeutic regimen for the patient all or a portion thereof, which supplies a menu driven application that presents to the patient the selected all or a portion of the configured therapist defined therapeutic regimen for the patient for execution by the patient;
executing the selected all or a portion of the configured therapist defined therapeutic regimen for the patient;
communicating status of the step of execution back to the central processing system of the selected all or a portion executed;
monitoring by the therapist via the therapist computer interface the status of the execution of the configured therapist defined therapeutic regimen for the patient and the portion thereof executed; and
inputting by the therapist via the therapist computer interface recommendations relating to the therapist defined therapeutic regimen for the patient relating to the selected therapist defined therapeutic regimen for the patient.

2. The method of claim 1, wherein the central processing system has associated therewith a plurality of templates to allow the therapist to create therapeutic regimens and the step of configuring by the therapist utilizes such templates.

3. The method of claim 1, wherein the central processing system is a web accessed system.

4. The method of claim 1, wherein the central processing system is contained on a computing device associated with the therapist computer interface.

5. The method of claim 1, and further comprising the step of creating a profile of the patient on the central processing system.

6. The method of claim 1, and further comprising the step of modifying the therapist defined therapeutic regimen for a patient during and in response to the step of monitoring.

7. The method of claim 1, wherein the therapist is able to monitor the execution of a therapist defined therapeutic regimen for a particular patient in real time and recommend modifications thereto in real time.

8. A method of attempting to help a person successfully complete physical therapy, comprising:
providing, via a first computing device including at least tangible computing elements, options for one or more physical therapy exercises to the first person;
providing, via the first computing device, instructions for performing the physical therapy exercises to the first person;
receiving, by the first computing device, information from the first person about performance of the physical therapy exercises; and
transmitting the information from the first computing device to a second computing device used by a second person.

9. A method as in claim 8, wherein the options for the physical therapy exercises comprise selecting from among possible exercises.

10. A method as in claim 9, wherein the possible exercises are arranged for selection in categories.

11. A method as in claim 10, wherein each of the categories contains exercises for a particular body part.

12. A method as in claim 8, wherein the options for a physical therapy exercise comprise one or more of time for each repetition of the exercise, a number of repetitions in a set for the exercise, a number of sets for the exercise, and a rest time between each of the sets for the exercise.

13. A method as in claim 8, wherein the steps further comprise recording the information about the performance of the physical therapy exercises over time.

14. A method as in claim 13, wherein the steps further comprise providing the information about the performance of the physical therapy exercises over time to the first person.

15. A method as in claim 13, wherein the step of transmitting the information from the first computing device to the second computing device further comprises transmitting the information about the performance of the physical therapy exercises over time to the second computing device.

16. A method as in claim 8, wherein the information is transmitted from the first computing device to the second computing device through one or more other computing devices, one or more networks, or both.

17. A method as in claim 8, wherein the information is transmitted from the first computing device to the second computing device via the Internet.
